(19) United States
(12) Patent Application Publication
(10) Pub. No.: US 2026/0224514 A1
(43) Pub. Date: Aug. 6, 2026

(54) SITE-SPECIFIC ACTIVATION OF REGULATORY T CELLS

(71) Applicant: MIGAL GALILEE RESEARCH INSTITUTE LTD., Kiryat Shmona (IL)

(72) Inventors: Tom WICKHAM, Scituate, MA (US); Chandra PATEL, Boston, MA (US); Gideon GROSS, Moshav Almagor (IL); Hadas WEINSTEIN-MAROM, Kibbutz Dafna (IL)

(21) Appl. No.: 19/111,661

(22) PCT Filed: Sep. 15, 2023
(86) PCT No.: PCT/US2023/032868

§ 371 Date: Mar. 13, 2025

Related U.S. Application Data
(60) Provisional application No. 63/407,103, filed on Sep. 15, 2022.

(51) Int. Cl. A61K 31/17; A61K 40/11; A61K 40/31; A61P 1/00; C07K 14/705; C12N 15/87

(52) U.S. Cl. CPC A61K 31/17; A61K 40/11; A61K 40/31; A61P 1/00; C07K 14/7051; C12N 15/87; A61K 2239/28

(57) ABSTRACT

Described herein are compositions and methods for producing immune cells, such as regulatory T cells (Tregs), expressing a chimeric antigen receptor (CAR) which responds to a non-degradable engineered polymeric antigen in a site-specific manner. Upon activation, the engineered immune cells have an immunosuppressive phenotype.

### REFERENCE TO RELATED APPLICATION

This application claims the benefit under 35 U.S.C. § 119(e) of U.S. Provisional Application No. 63/407,103, entitled “SITE-SPECIFIC ACTIVATION OF REGULATORY T CELLS,” filed Sep. 15, 2022, the contents of which are incorporated herein by reference in their entirety.

### BACKGROUND

Harnessing regulatory T cells (Tregs) for suppressing local inflammation and restoring immunological balance holds great promise in the treatment of numerous diseases including autoimmune disease, allergic disease, and inflammatory disease. Initiating or maintaining cell signaling to achieve a Treg phenotype using a chimeric antigen receptor (CAR), would allow for controlling a Treg immunosuppressive phenotype. As Tregs form only a minor fraction of the T cell population, there is need for effective therapies that can increase circulating Tregs under controlled conditions allowing for the treatment of autoimmune and autoimmune-related disorders.

### SUMMARY OF THE INVENTION

The present disclosure relates, at least in part, to methods and compositions for engineering immune cells (e.g., regulatory T cells) to express a chimeric antigen receptor (CAR), such as an anti-antigen CAR. The anti-antigen CAR is activated by a non-degradable engineered polymeric antigen inducing an immunosuppressive phenotype in regulatory T cells (Tregs).

The disclosure, in some aspects, provides a method for site-specific activation of engineered T cells present in a subject, the method comprising administering to the subject a composition comprising a non-degradable engineered polymeric antigen, wherein the engineered T cells comprise a chimeric antigen receptor (CAR) specific to the antigen.

In some embodiments, the method further comprises administering to the subject the engineered T cells.

The disclosure, in some aspects, provides a method for site-specific activation of engineered T cells in a subject, the method comprising administering to the subject engineered T cells comprising a CAR specific to a non-degradable engineered polymeric antigen. In some embodiments, the method further comprises administering to the subject the non-degradable engineered polymeric antigen. In some embodiments, the non-degradable engineered polymeric antigen is non-toxic. In some embodiments, the non-degradable engineered polymeric antigen retains at least 80% capacity to bind with the CAR one day after administering to the subject.

In some embodiments, the subject has gastrointestinal (GI) tract permeability wherein the non-degradable engineered polymeric antigen is absorbed into the gastrointestinal tract barrier at a site of breach of the barrier or local increase in permeability of the barrier (e.g., leaky places in the mucosal gut barrier) but does not get absorbed into the blood stream. In some embodiments, the non-degradable engineered polymeric antigen is not absorbed into the gastrointestinal tract barrier or into the blood stream in the absence of a breach in the gastrointestinal tract.

In some embodiments, the non-degradable engineered polymeric antigen comprises a polyethylene glycol (PEG), dextran, ethylene glycol, polyethylene oxide, poly(1-caprolactone) (PCL), polyglycolic acid (PGA), polylactic acid (PLA), and their copolymer poly(glycolic acid) (PLGA), polyvinyl alcohol (PVA), polyurethane (PU), collagen, fibrin, fibrinogen, gelatin, silk, elastin, myosin, keratin, actin, chitin, chitosan, alginate, hyaluronic acid, cellulose, agarose, and glycosaminoglycan. In some embodiments, the non-degradable engineered polymeric antigen comprises a polyethylene glycol (PEG). In some embodiments, the non-degradable engineered polymeric antigen comprises dextran. In some embodiments, the engineered polymeric antigen has a molecular weight of 400-10,000 g/mol.

In some embodiments, the subject has or is suspected of having a disease selected from an inflammatory or autoimmune disease. In some embodiments, the inflammatory condition is pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GVHD), tolerance induction for transplantation, transplant rejection, or sepsis. In some embodiments, the subject has or is at risk of developing inflammatory bowel. In some embodiments, the autoimmune disease is eczema, vitiligo, scleroderma, psoriasis, vasculitis, polymyalgia rheumatica, fibromyalgia, type 1 diabetes mellitus, multiple sclerosis, lupus, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, bullous pemphigoid, pemphigus vulgaris, autoimmune hepatitis, Sjogren's syndrome, celiac disease, central nervous system vasculitis, autoimmune-related epilepsy, Hashimoto's encephalopathy, steroid-responsive encephalopathy, neuromyelitis optica, optic neuritis, neurosarcoidosis, or neuro-Behcet's disease. In some embodiments, the disease is inflammatory bowel disease, Crohn's disease, or colitis.

In some embodiments, the composition is administered to the subject through oral ingestion or through direct injection at the site of site-specific activation. In some embodiments, the site of site-specific activation is comprised within the GI tract, a joint, a visceral organ, a muscle, the skin, the brain, the spine, or the CSF.

In some embodiments, the engineered T cells are CD3+ T cells, CD4+ T cells, or CD8+ T cells. In some embodiments, the engineered T cells are regulatory T cells. In some embodiments, the regulatory T cells are engineered to stably express FoxP3, IL10, or IL2.

In some embodiments, the CAR comprises an extracellular antigen binding domain comprising a sequence that binds to a non-degradable engineered polymeric antigen; a signaling domain; and a co-stimulatory domain. In some embodiments, the extracellular domain comprises a polypeptide comprising an antibody, antibody fragment, antigen-binding domain of an antibody (e.g., single chain antibodies, Fab and sFab fragments, F(ab′)2, Fd fragments, Fv fragments, scFv, CDRs, and domain antibody (dAb) fragments), immunoglobulin variable domain, or immunoglobulin variable domain sequence. In some embodiments, the signaling domain comprises a domain of any one of TCRzeta, FcRgamma, FcRbeta, CD3gamma, CD3theta, CD3delta, CD3epsilon, CD3zeta, CD22, CD79a, CD79b, or CD66dCD3zeta. In some embodiments, the co-stimulatory domain comprises a domain of any one of CARD 11, CD2, CD7, CD27, CD28, CD30,CD40,CD54(ICAM), CD83,CD134(OX40), CD137(4-1BB), CD150 (SLAMF1), CD152(CTLA4), CD223 (LAG3), CD270(HVEM), CD273 (PD-L2), CD274 (PD-L1), CD278 (ICOS), DAP10, LAT, NKD2C SLP76, TRIM, or ZAP70. In some embodiments, the signaling domain comprises the domain of CD3zeta and the co-stimulatory domain comprises the domain of CD28. In some embodiments, the signaling domain comprises the domain of CD3zeta and the co-stimulatory domain comprises the domain of CD137.

In some embodiments, the subject has or is suspected of having an inflammatory bowel disease, the engineered T cells comprises a CAR comprising: an extracellular antigen binding domain comprising a sequence that binds to a non-degradable engineered polymeric antigen; a signaling domain of CD3zeta; and a co-stimulatory domain of CD28, or CD137; the administration of the non-degradable engineered polymeric antigen is through oral ingestion, the site of site-specific activation is the GI tract, and the non-degradable engineered polymeric antigen is a PEG having a molecular weight of 400-10,000 g/mol.

The disclosure, in some aspects, provides a method for activation of engineered T cells present in the GI tract of a subject having or suspected of having an inflammatory bowel disease, the method comprising orally administering to the subject, a composition comprising a non-degradable engineered PEG antigen having a molecular weight of 400-10,000 g/mol, wherein the engineered T cells comprise a CAR comprising: an extracellular antigen binding domain comprising a sequence that binds to the non-degradable engineered polymeric antigen; a signaling domain of CD3zeta; and a co-stimulatory domain of CD28, or CD137.

In some embodiments, the method further comprises administering to the subject the engineered T cells. In some embodiments, the composition is administered to the subject on a daily, weekly, or monthly basis.

In some embodiments, the engineered T cells are CD3+ T cells, CD4+ T cells, or CD8+ T cells. In some embodiments, the engineered T cells are regulatory T cells. In some embodiments, the regulatory T cells are engineered to stably express FoxP3, IL10, or IL2.

The disclosure, in some aspects, provides a non-degradable engineered polymeric antigen comprising a domain, wherein the non-degradable engineered polymeric antigen is a polyethylene glycol (PEG) or dextran, the non-degradable engineered polymeric antigen is formulated in an injectable or ingestible form, the non-degradable engineered polymeric antigen is engineered to have a molecular weight between 200 and 50,000 g/mol, the non-degradable engineered polymeric antigen is engineered to have a molecular weight between 400 and 20,000 g/mol, the non-degradable engineered polymeric antigen is administered to a subject following administration to the subject of an engineered T cell, wherein the engineered T cell expresses a chimeric antigen receptor (CAR) comprising an extracellular antigen binding domain comprising a sequence that binds to the non-degradable engineered polymeric antigen.

The disclosure, in some aspects, provides a method of making a non-degradable polymeric antigen comprising polymerizing a biocompatible, non-toxic, inert monomer in a chemical process. In some embodiments, the method further comprising formulating the non-degradable polymeric antigen with a pharmaceutically acceptable carrier to produce a composition for administration to a subject.

The disclosure, in some aspects, provides a nucleic acid comprising a nucleotide sequence encoding a chimeric antigen receptor (CAR) comprising: an extracellular antigen binding domain comprising a sequence that binds to a non-degradable engineered polymeric antigen; a signaling domain; and a co-stimulatory domain. In some embodiments, the extracellular domain comprises a polypeptide comprising an antibody, antibody fragment, antigen-binding domain of an antibody (e.g., single chain antibodies, Fab and sFab fragments, F(ab′)2, Fd fragments, Fv fragments, scFv, CDRs, and domain antibody (dAb) fragments), immunoglobulin variable domain, or immunoglobulin variable domain sequence. In some embodiments, the signaling domain comprises a domain of any one of TCRzeta, FcRgamma, FcRbeta, CD3gamma, CD3theta, CD3delta, CD3epsilon, CD3zeta, CD22, CD79a, CD79b, or CD66dCD3zeta. In some embodiments, the co-stimulatory domain comprises a domain of any one of CARD 11, CD2, CD7, CD27, CD28, CD30, CD40,CD54(ICAM), CD83,CD134(OX40), CD137(4-1BB), CD150 (SLAMF1), CD152(CTLA4), CD223 (LAG3), CD270(HVEM), CD273(PD-L2), CD274 (PD-L1), CD278 (ICOS), DAP10, LAT, NKD2C SLP76, TRIM, or ZAP70. In some embodiments, the signaling domain comprises the domain of CD3zeta and the co-stimulatory domain comprises the domain of CD28. In some embodiments, the signaling domain comprises the domain of CD3zeta and the co-stimulatory domain comprises the domain of CD137.

In some embodiments, the non-degradable engineered polymeric antigen is a polyethylene glycol or dextran.

In some embodiments, an engineered T cell engineered by contacting a T cell with any one on the nucleic acids described herein.

The disclosure, in some aspects, provides an engineered T cell comprising a chimeric antigen receptor (CAR) comprising: an extracellular antigen binding domain comprising a sequence that binds to a non-degradable engineered polymeric antigen; a signaling domain of CD3zeta; and a co-stimulatory domain of CD28, or CD137. In some embodiments, the engineered T cell is a CD3+ T cell, CD4+ T cell, or CD8+ T cell. In some embodiments, the engineered T cell is a regulatory T cell. In some embodiments, the regulatory T cell is engineered to stably express FoxP3, IL10, or IL2. In some embodiments, the cell is obtained from a donor. In some embodiments, the cell is obtained from an individual having or suspected of having an autoimmune disease.

In some embodiments, the cell is administered to an individual having or suspected of having an autoimmune disease. In some embodiments, the disease is selected from an inflammatory bowel disease, Crohn's disease, celiac disease, rheumatoid arthritis, diabetes, lupus, eczema, vitiligo, scleroderma, psoriasis, vasculitis, polymyalgia rheumatica, fibromyalgia, autoimmune encephalitis, multiple sclerosis, central nervous system vasculitis, autoimmune-related epilepsy, Hashimoto's encephalopathy, steroid-responsive encephalopathy, neuromyelitis optica, optic neuritis, neurosarcoidosis, or neuro-Behcet's disease.

In some embodiments, the non-degradable engineered polymeric antigen is a polyethylene glycol or dextran.

The disclosure, in some aspects, provides a method comprising contacting a cell with a nucleic acid comprising a nucleotide sequence encoding a chimeric antigen receptor (CAR) comprising an extracellular antigen binding domain comprising a sequence that binds to a non-degradable engineered polymeric antigen. In some embodiments, the non-degradable engineered polymeric antigen is a polyethylene glycol or dextran.

In some embodiments, the CAR further comprises: a signaling domain of CD3zeta; and a co-stimulatory domain of CD28, or CD137.

In some embodiments, the nucleic acid is introduced in the cell in vitro via a transfection, a transduction, or a gene editing technique. In some embodiments, the nucleic acid is delivered to the cell through a viral or non-viral vector. In some embodiments, the nucleic acid is delivered to the cell through a viral vector. In some embodiments, the nucleic acid is delivered to the cell through a non-viral vector.

In some embodiments, the viral vector is selected from a modified virus derived from a virus selected from the group consisting of a retrovirus, lentivirus, gammavirus, adenovirus, adeno-associated virus, poxvirus, alphavirus, and herpes virus. In some embodiments, the non-viral vector is a polymer nanoparticle, lipid, calcium phosphate, DNA-coated microparticle, or transposon.

In some embodiments, the engineered T cell expresses the CAR.

The details of one or more embodiments of the invention are set forth in the description below. Other features or advantages of the present invention will be apparent from the following drawings and detailed description of several embodiments, and also from the appended claims.

### DETAILED DESCRIPTION

It is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited in the present application are incorporated by reference for the purposes or subject matter referenced in this disclosure.

### Introduction

Provided herein are the compositions and methods for engineering immune cells (e.g., regulatory T cells) to express a chimeric antigen receptor (CAR), such as an anti-antigen CAR. Also provided herein are methods of administration of the CAR immune cells and use as a treatment. In some embodiments, a non-degradable engineered polymeric antigen (such as polyethylene glycol (PEG) or dextran) is administered to the subject in order to activate anti-non-degradable engineered polymeric antigen CAR immune cells in a site-specific manner. In some embodiments, the anti-antigen CAR immune cells may be used to treat autoimmune and inflammatory diseases.

### Engineered Immune Cells

Embodiments of the disclosure include engineered immune cells, for example, engineered regulatory T cells (Tregs). Non-limiting examples of immune cells include B cells, T cells, and natural killer (NK) cells. In some embodiments, the methods provided herein comprise editing CD3+ cells, thereby producing edited CD3+ cells, including CD4+ and CD8+ Treg cells. In some embodiments, the methods comprise editing CD4+ T cells, thereby producing CD4+ Treg cells. In some embodiments, the methods comprise editing CD8+ T cells, thereby producing CD8+ Treg cells. In some embodiments, the methods comprise editing NK1.1+ T cells, thereby producing NK1.1+ Treg cells.

In some embodiments, the methods comprise editing a stem cell. In some embodiments, the methods comprise editing a pluripotent stem cell. In some embodiments, the methods comprise editing CD34+ hematopoietic stem cells (HSCs). In some embodiments, the methods comprise editing induced pluripotent stem cells (iPSCs). Edited stem cells may be matured in vitro to produce Treg cells, or administered to a subject to allow in vivo development into Treg cells. Edited stem cells may be matured into CD3+ Treg cells, CD4+ Treg cells, CD8+ Treg cells, NK1.1+ Treg cells, or a combination thereof.

In some embodiments, a method comprises editing a T cell. A T cell or T lymphocyte is an immune system cell that matures in the thymus and produces a T cell receptor (TCR), e.g., an antigen-specific heterodimeric cell surface receptor typically comprised of an α-β heterodimer or a γ-δ heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7, CD28, CD3, CD127, and CD45RA, and decreased or no expression of CD45RO as compared to TCM), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+ CD25+ (FoxP3+) regulatory T cells and Treg17 cells, as well as Tr1, Th3, CD8+CD28−, or Qa-1 restricted T cells. In some embodiments, the cell is a CD3+, CD4+, and/or CD8+ T cell. In some embodiments, the cell is a CD3+ T cell. In some embodiments, the cell is a CD4⁺CD8⁻ T cell. In some embodiments, the cell is a CD4^{-CD}8⁺ T cell. In some embodiments, the cell is a regulatory T cell (Treg). Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Ra on its surface.

In some embodiments, the T cells are obtained from a donor. In some embodiments, the T cells are obtained from a subject having or suspected of having an autoimmune disease or inflammatory disease.

In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood. In some embodiments, the cell is obtained by sorting cells of peripheral blood to attain a desired cell population (e.g., CD3+ cells), and one or more cells of the sorted population are modified by a method described herein. Also contemplated herein are cells produced by a method described herein.

Embodiments of genetically modified cells described herein may be any cell type known in the art. In some embodiments, the cell is a T cell, a precursor T cell, or a hematopoietic stem cell. In some embodiments, the cell is an NK-T cell (e.g., a FoxP3−NK-T cell or a FoxP3+NK-T cell). In some embodiments, the cell is a regulatory B (Breg) cell (e.g., a FoxP3−B cell or a FoxP3+B cell). In some embodiments, the cell is a CD4+ T cell (e.g., a FoxP3−CD4+ T cell or a FoxP3+CD4+ T cell) or a CD8+ T cell (e.g., a FoxP3−CD8+ T cell or a FoxP3+CD8+ T cell). In some embodiments, the cell is a CD25− T cell. In some embodiments, the cell is a regulatory T (Treg) cell. Non-limiting examples of Treg cells are Tr1, Th3, CD8+CD28−, and Qa-1 restricted T cells. In some embodiments, the Treg cell is a FoxP3+ Treg cell. In some embodiments, the Treg cell expresses CTLA-4, LAG-3, CD25, CD39, CD27, CD70, CD357 (GITR), neuropilin-1, galectin-1, and/or IL-2Ra on its surface.

In some embodiments, the cell is a human cell. In some embodiments, a cell as described herein is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood. In some embodiments, the cell is obtained by sorting cells of peripheral blood to attain a desired cell population (e.g., CD3+ cells), and one or more cells of the sorted population are modified by a method described herein. Also contemplated herein are cells produced by a method described herein.

In some embodiments, the immune cell comprises a T cell. The type of T cell selected is of importance for successful clinical implementation. T cells are immune cells with two major subsets, CD8+ and CD4+, that play a role in adaptive immune response. Tregs are a specialized subpopulation of T cells that are CD4+, suppress the immune response, with the ability to inhibit T cell proliferation and cytokine production. There is compelling evidence that engagement of Tregs with antigen through T cell receptors (TCR) is critical for immune suppression. Tr1 cells are a subset of CD4(+) FoxP3(+/−) Tregs which are induced in the periphery in an T cell receptor-(TCR) and antigen-specific manner upon chronic exposure to antigen on dendritic cells. These cells are characterized by a non-proliferative (anergic) state, high production of IL10 and Transforming growth factor β (TGF-β) but only minimally of IL-2 and none of IL-4 or IL-17 and the ability to suppress effector T cells (Teffs) in a cell-to-cell contact-independent manner; therefore, suppressing inflammation.

In some embodiments the methods and compositions provided herein relate to engineering CD3+ T cells, CD4+ T cells, or CD8+ T cells into Tregs or Tr1 cells, stably expressing FOXP3, IL10, or IL2. “Stably expressed” characterizes long term gene expression that gets passed on to future generations of the cell. FOXP3 functions as a regulator in the regulatory pathway for Tregs, IL10 is an anti-inflammatory cytokine, and IL2 is a cytokine that regulates immunity. Major sources of IL10 and IL2 are CD4+ and CD8+ T cells.

In some embodiments, an immune cell (e.g., a T cell) expresses a chimeric antigen receptor (CAR). A CAR refers to an artificial T cell receptor or a genetically engineered receptor, which grafts the desired specificity onto an immune cell. In some embodiments, the genetically engineered cell further comprises a sequence that encodes a CAR. In some embodiments, the disclosure provides a therapeutic cell. As used herein, a “therapeutic cell,” refers to an engineered immune cell, e.g., an engineered regulatory T cell, expressing a chimeric antigen receptor. A CAR, in some embodiments, binds to a non-degradable engineered polymeric antigen (for example, to direct the therapeutic cell (e.g., an engineered Treg cell) to a target cell or tissue). In some embodiments, the therapeutic cell is a Treg.

Thus, in one aspect, the present disclosure provides a nucleic acid molecule comprising a nucleotide sequence encoding a CAR. In some embodiments, the CAR comprises an extracellular antigen binding domain (ectodomain), a signaling domain, and an intracellular domain (e.g., comprising a co-stimulatory domain). In some embodiments, the extracellular antigen binding domain comprises a polypeptide that binds to a non-degradable engineered polymeric antigen, as described herein. In some embodiments, the polypeptide comprises an antibody, antibody fragment, antigen-binding domain of an antibody (e.g., single chain antibodies, Fab and sFab fragments, F(ab′)2, Fd fragments, Fv fragments, scFv, CDRs, and domain antibody (dAb) fragments), immunoglobulin variable domain, or immunoglobulin variable domain sequence. In some embodiments, the signaling domain is any one of TCRzeta, FcRgamma, FcRbeta, CD3gamma, CD3theta, CD3delta, CD3epsilon, CD3zeta, CD22, CD79a, CD79b, or CD66dCD3zeta. In some embodiments, the co-stimulatory domain is any one of CARD 11, CD2, CD7, CD27, CD28, CD30, CD40, CD54(ICAM), CD83, CD134(OX40), CD137 (4-1BB), CD150 (SLAMF1), CD152(CTLA4), CD223 (LAG3), CD270 (HVEM), CD273 (PD-L2), CD274 (PD-L1), CD278 (ICOS), DAP10, LAT, NKD2C SLP76, TRIM, or ZAP70. In some embodiments, the signaling domain comprises a CD3zeta domain and the co-stimulatory domain comprises the CD28 or CD137 domain.

### Non-Degradable Engineered Polymeric Antigen

To activate the anti-antigen CAR in a subject, a non-degradable, non-toxic, engineered polymeric antigen is administered. As used herein, “non-degradable” refers to a non-degradable engineered polymeric antigen wherein >80% of the non-degradable engineered polymeric antigen remains intact after 1 day of incubation with gut enzymes. As used herein, “non-toxic” refers to a substance that does not cause adverse events at less than 10 times the scaled dose used in animal safety and toxicity studies. Additionally, “non-toxic” refers to components that are already approved for use in humans or has been shown to be safe at doses anticipated to be used with engineered Tregs.

Non-limiting examples of non-degradable engineered polymeric antigens include but are not limited to, polyethylene glycol (PEG), dextran, ethylene glycol, polyethylene oxide, and, poly(1-caprolactone) (PCL), polyglycolic acid (PGA), polylactic acid (PLA), and their copolymer poly(glycolic acid) (PLGA), polyvinyl alcohol (PVA), polyurethane (PU), collagen, fibrin, fibrinogen, gelatin, silk, elastin, myosin, keratin, actin, chitin, chitosan, alginate, hyaluronic acid, cellulose, agarose, and glycosaminoglycans. In some embodiments, non-degradable engineered polymeric antigens are administered in combination; that is, in some embodiments, 2, 3, 4, 5 or more non-degradable engineered polymeric antigens are administered to a subject (for example, to active the same or different engineered immune cells).

PEG is a synthetic polymer derived from petroleum and used for many applications including medicine. PEG has the chemical structure of (CH₂CH₂O)n. PEG is highly flexible hydrophobic, non-ionic, and biocompatible, non-immunogenic, as PEG chains aid in drug solubility and stability. In addition, PEG is used as an excipient in pharmaceutical products and laxatives. Moreover, PEG can be crosslinked to act as an extracellular matrix. PEG is also known as polyethylene oxide (PEO) and polyoxyethlene (POE). The FDA has deemed PEG biologically inert and safe with over 20 PEGylated systems approved, and more in clinical trials.

PEG size is an important component to promote a balance between crosslinking and permeability. A variety of cross-linking methods are used with PEGs, the selected process can have an effect on the physiochemical characteristics of the PEG scaffold. In some embodiments the physiochemical characteristics include but are not limited to, permeability, molecular diffusion, elasticity, and rate of degradation. Commercially available PEG molecules range in size from 300 g/mol to 10,000,000 g/mol. In some embodiments, PEGS are available in different geometries; branched, star, and comb. In some embodiments branched PEGs have a central core with three to ten PEG chains branched off. In some embodiments a star peg has a central core, with 10 to 100 PEG chains branching off it. In some embodiments a comb PEG has a polymer backbone with multiple PEG chains attached. For activation and treatment, the PEG needs to be large enough to cross-link, increasing permeability and bind PEG-CAR, but not too large that it cannot diffuse through a barrier (e.g., GI tract) and bind to the anti-PEG CAR in a site-specific manner.

Dextran is a generic term for a family of polysaccharides derived from glucose or glucan, with a molecular formula of H(C₆H₁₀O₅)ₓOH. In some embodiments dextran consists of a polymer chain of α-1,6 glycosidic linkages between glucose monomers with α-1,3 linkages branching off. In some embodiments, dextran chains range from 3 to 2000 kilodaltons (kDa).

The non-degradable engineered polymeric antigen, such as PEG, is able to permeate inflamed tissue (e.g., in the GI tract). Permeability is the state of a membrane that allows molecules to pass through it. In some embodiments, the non-degradable engineered polymeric antigen is able to permeate the GI tract due to increased gut permeability. Gut permeability is the ability for molecules to pass through the junctions in the gut epithelial wall. Increased gut permeability involves increasing these junctions between the individual cells lining the gastrointestinal tract.

In some embodiments the molecular weight of the non-degradable engineered polymeric antigen is from 200 g/mol to 50,000 g/mol. In some embodiments, the non-degradable engineered polymeric antigen has a molecular weight from 200 to 300 g/mol. In some embodiments, the non-degradable engineered polymeric antigen will have a molecular eight from 300 to 400 g/mol. In some embodiments, the non-degradable engineered polymeric antigen will have a molecular weight from 400 to 500 g/mol. In some embodiments, the non-degradable engineered polymeric antigen will have a molecular weight from 500 to 600 g/mol. In some embodiments, the non-degradable engineered polymeric antigen will have a molecular weight from 600 to 700 g/mol. In some embodiments, the non-degradable engineered polymeric antigen will have a molecular weight from 700 to 800 g/mol. In some embodiments, the non-degradable engineered polymeric antigen will have a molecular weight from 800 to 900 g/mol. In some embodiments, the non-degradable engineered polymeric antigen will have a molecular weight from 900 to 1,000 g/mol. In some embodiments, the non-degradable engineered polymeric antigen will have a molecular weight from 1,000 to 2,000 g/mol. In some embodiments, the non-degradable engineered polymeric antigen will have a molecular weight from 2,000 to 3,000 g/mol. In some embodiments, the non-degradable engineered polymeric antigen will have a molecular weight from 3,000 to 4,000 g/mol. In some embodiments, the non-degradable engineered polymeric antigen will have a molecular weight from 4,000 to 5,000 g/mol. In some embodiments, the non-degradable engineered polymeric antigen will have a molecular weight from 5,000 to 6,000 g/mol. In some embodiments, the non-degradable engineered polymeric antigen will have a molecular weight from 6,000 to 7,000 g/mol. In some embodiments, the non-degradable engineered polymeric antigen will have a molecular weight from 7,000 to 8,000 g/mol. In some embodiments, the non-degradable engineered polymeric antigen will have a molecular weight from 8,000 to 9,000 g/mol. In some embodiments, the non-degradable engineered polymeric antigen will have a molecular weight from 9,000 to 10,000 g/mol. In some embodiments, the non-degradable engineered polymeric antigen will have a molecular weight from 10,000 to 20,000 g/mol. In some embodiments, the non-degradable engineered polymeric antigen will have a molecular weight from 20,000 to 30,000 g/mol. In some embodiments, the non-degradable engineered polymeric antigen will have a molecular weight from 30,000 to 40,000 g/mol. In some embodiments, the non-degradable engineered polymeric antigen will have a molecular weight from 40,000 to 50,000 g/mol.

In some embodiments the composition will be formulated in an injectable or ingestible form. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

### Generation of Engineered Cells

In some aspects, provided herein is a method of generating an immune cell comprising any one of the CARs provided herein. In some embodiments, a method of making such a cell comprises transfecting a cell with any one or more of the nucleic acids comprising polynucleotides encoding one or more polypeptides of a CAR as disclosed herein. Transfection may comprise the use of chemical reagents (e.g., a cationic polymer), lipids, or physical methods such as microinjection or electroporation. In some embodiments, transfection comprises contacting cells with a vector (e.g., viral or non-viral vectors) comprising a nucleic acid encoding a polypeptide of a CAR. In some embodiments compositions to administer may include nanoparticles that encode a CAR.

In some embodiments, the cells are T cells. In some embodiments, the T cells are CD3+, CD4+, and/or CD8+ T cells. In some embodiments, the T cells are engineered to be Treg cells (e.g., Tr1 cells). In some embodiments of producing Tregs (e.g., Tr1 cells) with stable suppressive function are performed in vivo by administering to a subject reagents/composition that induce chimeric antigen receptor (CAR) expression in cells (e.g., immune cells of the subject such as CD3+, CD4+ or CD8). In some embodiments, a gene encoding a CAR is expressed in a cell, e.g., by delivery of nucleic acids comprising the gene into a subject. In some embodiments, a method of producing Tregs or Tr1 cells with stable suppressive function in vivo comprises a CAR that induces Treg or Tr1 differentiation. Compositions to administer may include nucleic acids, e.g., comprised in vectors (e.g., viral or non-viral vectors) or formulated using nanoparticles, that encode the CAR.

Some embodiments of methods of producing Tregs with stable suppressive function are performed ex vivo, e.g., in isolated immune cells that are transfected with, contacted with, or treated with reagents/compositions that induce CAR expression in cells (e.g., immune cells of the subject such as CD3+, CD4+ or CD8+). Cells thus engineered are then administered to a subject. In some embodiments, a gene encoding a CAR is expressed in a cell, e.g., by delivery of nucleic acids comprising the gene into a subject. In some embodiments, a method of producing Tregs or Tr1 cells with stable suppressive function ex vivo comprises a CAR that induces Treg or Tr1 differentiation. In some embodiments, the T cells are obtained from a donor. In some embodiments, the T cells are obtained from a subject having or suspected of having an autoimmune disease or inflammatory disease.

Some aspects of the disclosure relate to compositions for administering into a subject so that they target particular cells (e.g., immune cells) as to be engineered into Treg or Tr1 cells in vitro. Such compositions may include nucleic acids, e.g., comprised in vectors (e.g., viral or non-viral vectors) or formulated using nanoparticles, that encode chimeric antigen receptors (CARs) that are activated by non-degradable engineered polymeric antigen. In some embodiments, a composition for making Tregs or Tr1 cells according to the methods disclosed herein comprises one or more nucleic acids encoding CARs that induces differentiation and enhance suppressive function. In some embodiments, a method of producing Tregs or Tr1 cells with stable suppressive function in vitro comprises a CAR that induces Treg or Tr1 differentiation.

Methods for isolating and preparing T cells are well known in the art and often rely on commercial kits and protocols from leading companies in this field. Some examples for commercial kits include, but are not limited to:
1. ThermoFisher Scientific: Isolation of Untouched Human CD4+ T Cells from Peripheral Blood Mononuclear Cells (PBMC):
   thermofisher.com/il/en/home/references/protocols/proteins-expression-isolation-and-analysis/ cell-separation-methods/human-cell-separationprotocols/isolation-of-untouched-human-cd4-t-cells.html;
2. Miltenyi Biotec: CD 4+ T Cell Isolation Kit, human; mil ten yibiotec.com/CA-en/products/macs-cell-separation/cell-separationreagents/microbeads-and-isolation-kits/t-cells/ cd4-t-cell-isolation-kit-human.html;
3. STEMCELL Technologies: EasySep™ Human CD 4+ T Cell Isolation Kit; stemcell.com/easysep-human-cd4-t-cell-isolation-kit.html; and
4. BD Biosciences: Human Naive CD4 T Cell Enrichment Set bdbiosciences.com/ eu/reagents/research/magnetic-cell-separation/humancell-separation-reagents/human-naive-cd4-t-cell-enrichment-set - - - dm/p/558521.

In some embodiments, a nucleic acid described herein (e.g., for introduction into a cell or administration to a subject) is comprised in a vector. The term “vector” is used to refer to any molecule (e.g., nucleic acid, plasmid) or arrangement of molecules (e.g., virus) used to transfer coding information to a host cell. The term “expression vector” refers to a vector that is suitable for introduction of a host cell and contains nucleic acid sequences that direct and/or control expression of introduced heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the methods provided herein are lentiviral vectors. In some embodiments, one or more vectors are adenoviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors. In some embodiments, one or more AAV vectors are AAV9 vectors.

In some embodiments non-viral approaches include, but are not limited to, polymer nanoparticles, lipids, lipid nanoparticles, liposomes, calcium phosphate, transposon, electroporation/nucleofection or biolistic delivery of DNA-coated microparticles (Matuskova and Durinikova, 2016. Advances in Molecular Retrovirology InTech).

As will be understood by those skilled in the art, nucleic acids may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person. In some embodiments nucleic acids may be from a donor template.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of or about of 10,000, 5000, 3000, 2,000, 1,000, 500, 200,100, or 50 base pairs in length, and the like, (including all intermediate lengths) are contemplated to be useful.

When comparing polynucleotide or nucleic acid sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

Some aspects of the disclosure relate to methods of producing a genetically modified cell by inserting a heterologous promoter into a nucleic acid of a cell genome (e.g., by homologous recombination) upstream from a coding sequence on the nucleic acid, such that the inserted promoter becomes operably linked to a coding sequence of the endogenous gene. In some embodiments, a donor template comprising the heterologous promoter is introduced into the cell and incorporated into the genome by homologous recombination.

In some embodiments, a donor template comprising the heterologous promoter is introduced into the cell and incorporated into the genome by homologous recombination. In some embodiments, the donor template comprising the heterologous promoter further comprises a homology arm comprising a modified coding sequence or portion thereof, such that integration of the donor template into the cell genome replaces the codons encoding the substituted amino acids with codons encoding the desired amino acids. In some embodiments, the homology arm comprising the modified coding sequence or portion thereof comprises a homologous nucleic acid sequence downstream from the modified coding sequence or portion thereof, where the homologous nucleic acid sequence is identical to an endogenous sequence downstream from the endogenous coding sequence to be modified, to promote homologous recombination.

In some embodiments, the endogenous coding sequence is modified before insertion of the heterologous promoter. In some embodiments, the endogenous coding sequence is modified after insertion of the heterologous promoter (e.g., by incorporation of a second donor template by a second homologous recombination event).

Aspects of the disclosure relate to the use of nucleases to introduce a double-stranded break into nucleic acid of a cell genome and edit the genome at a desired locus (e.g., to promote integration of a donor template at the locus by homology-directed repair and/or inactivate a targeted gene). Any one of a number of gene- or genome-editing methods can used to accomplish editing of one or more loci. Non-limiting examples of gene editing methods include use of a DNA endonuclease such as an RNA-guided nuclease (e.g., Cas (e.g., Cas9) nuclease), zinc finger nuclease (ZFN), transcription activator-like effector nuclease (TALEN), or meganuclease; transposon-mediated gene editing; serine integrase-mediated gene editing; and lentivirus-mediated gene editing. In some embodiments, a gene editing method comprises knocking out or inactivating an endogenous gene, such as by producing a chromosomal gene knockout in the genome. As used herein, the term “chromosomal gene knockout” refers to a genetic alteration, inactivation, or introduced inhibitory agent in a host cell that prevents (e.g., reduces, delays, suppresses, or abrogates) production, by the host cell, of a functionally active endogenous polypeptide product. Alterations resulting in a chromosomal gene knockout or inactivation can include, for example, introduced nonsense mutations (including the formation of premature stop codons), missense mutations, gene deletion, or strand breaks.

In certain embodiments, a chromosomal gene knock-out or gene knock-in (e.g., insertion) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. A DNA endonuclease refers to an endonuclease that is capable of catalyzing cleavage of a phosphodiester bond within a DNA polynucleotide. In certain embodiments, an endonuclease is capable of cleaving a nucleic acid sequence in a targeted gene, thereby inactivating or “knocking out” the targeted gene. In some embodiments, an endonuclease is capable of cleaving a nucleic acid sequence in a targeted locus, promoting insertion of an exogenous nucleic acid sequence into the targeted locus by homologous recombination. An endonuclease may be a naturally occurring, recombinant, genetically modified, or fusion endonuclease. Examples of endonucleases for use in gene editing include zinc finger nucleases (ZFN), TALE-nucleases (TALEN), CRISPR-Cas nucleases, meganucleases, or megaTALs.

The nucleic acid strand breaks caused by DNA endonucleases are typically double-strand breaks (DSB), which may be commonly repaired through the distinct mechanisms of homology directed repair (HDR) by homologous recombination, or by non-homologous end joining (NHEJ). (NHEJ: Ghezraoui et al., 2014 Mol Cell 55(6): 829-842; HDR: Jasin and Rothstein, 2013 Cold Spring Harb Perspect Biol 5(11): a012740, PMID 24097900) During HDR/homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. HDR is favored by the presence of a donor template at the time of DSB formation.

As used herein, a “zinc finger nuclease” (ZFN) refers to a fusion protein comprising a zinc finger DNA-binding domain fused to a non-specific DNA cleavage domain, such as a Fok1 endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90: 2256-2260, 1993; Wolfe et al., J. Mol. Biol. 285: 1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair (HDR). Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout or inactivation comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little, or no sequence overlap for annealing, thereby introducing errors that knock out gene expression. Alternatively, homology directed repair (HDR) can introduce a transgene at the site of DSB providing homologous flanking sequences are present in the donor template containing the transgene. In certain embodiments, a gene knockout comprises an insertion, a deletion, a mutation, or a combination thereof, and made using a TALEN molecule.

Gene-editing systems and methods described herein may make use of viral or non-viral vectors or cassettes, as well as nucleases that allow site-specific or locus-specific gene-editing, such as RNA-guided nucleases, Cas nucleases (e.g., Cpf1 or Cas9 nucleases), meganucleases, TALENs, or ZFNs. Certain RNA-guided nucleases useful with some embodiments provided herein are disclosed in U.S. Pat. No. 11,162,114, which is expressly incorporated by reference herein in its entirety. Non-limiting examples of Cas nucleases include SpCas9, SaCas9, CjCas9, xCas9, C2c1, Cas13a/C2c2, C2c3, Cas13b, Cpf1, and variants thereof. Certain features useful with some embodiments provided herein are disclosed in WO 2019/210057, which is expressly incorporated by reference in its entirety.

As used herein, a “clustered regularly interspaced short palindromic repeats/Cas” (CRISPR/Cas, or Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into types (e.g., type I, type II, type III, and type V) based on the sequence and structure of the Cas nucleases. The crRNA-guided surveillance complexes in types I and III need multiple Cas subunits. The Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA: tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a donor template transgene with homologous flanking sequences can be introduced at the site of DSB via homology directed repair (HDR). The crRNA and tracrRNA can be engineered into a single guide RNA (sgRNA or gRNA) (see, e.g., Jinek et al., Science 337: 816-21, 2012). Further, the region of the guide RNA complementary to the target site can be altered or programed to target a desired sequence (Xie et al., PLOS One 9: e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). Non-limiting examples of CRISPR/Cas nucleases include Cas9, SaCas9, CjCas9, xCas9, C2C1, Cas13a/C2c2, C2c3, Cas13b, Cpf1, and variants thereof.

In some embodiments, a gene knockout or inactivation comprises an insertion, a deletion, a mutation or a combination thereof, and made using an RNA-guided nuclease. Exemplary gRNA sequences and methods of using the same to knock out endogenous genes that encode immune cell proteins include those described in Ren et al., Clin Cancer Res. 2017. 23(9): 2255-2266, the gRNAs, Cas9 DNAs, vectors, and gene knockout techniques of which are hereby expressly incorporated by reference in their entirety.

In some embodiments, a gene modification comprises an insertion of an exogenous nucleic acid sequence (e.g., heterologous promoter, transgene, and/or combinations thereof) into the genome of a cell, where an RNA-guided nuclease introduces a double-stranded break in the genome and the exogenous nucleic acid sequence is introduced into the genome by homology-directed repair.

In some embodiments, the CAR is expressed episomally in a cell. Episomal expression may be achieved by any method known in the art, such as delivery of an RNA (e.g., mRNA or self-amplifying RNA) or DNA (e.g., plasmid or artificial chromosome) encoding the CAR.

The disclosure, in some aspects, provides the use of a heterologous promoter to express a CAR or portion thereof. The heterologous promoter may be any promoter known in the art. In some embodiments, the heterologous promoter is a constitutive promoter. In some embodiments, the promoter is an EF-1α, a PGK promoter, or an MND promoter. In some embodiments, the promoter is an MND promoter.

### Stabilized FoxP3 Expression

Some embodiments of methods of modifying cells described herein comprise introducing a genetic modification in a cell that stabilizes expression of FoxP3. Similarly, some embodiments of cells described herein comprise a genetic modification that stabilizes or increases FoxP3expression, relative to an unmodified cell. Additionally, some embodiments of nucleic acids and vectors described herein stabilize FoxP3 expression in a cell.

In some embodiments, an endogenous FOXP3 locus is modified in a cell, resulting in stabilized expression. For example, in some embodiments, a heterologous promoter is inserted within or downstream from a Treg-specific demethylated region (TSDR) in the genome, and upstream from a first coding exon of an endogenous FOXP3 coding sequence. In some embodiments, a promoter is inserted downstream from the TSDR, and within or upstream from the first coding exon of FOXP3. Insertion of a heterologous promoter in this manner bypasses endogenous regulation of FOXP3 by the TSDR, which can become methylated in inflammatory conditions, inhibiting transcription of the endogenous FOXP3 coding sequence from the endogenous FOXP3 promoter located upstream from the TSDR. Thus, such stabilized FoxP3 expression by heterologous promoter insertion allows stable FoxP3 expression even in inflammatory conditions, preventing transdifferentiation into a T effector cell.

The heterologous promoter may be inserted at any position between the endogenous promoter and the first coding exon of the FOXP3 coding sequence. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides downstream from the TSDR of FOXP3. In some embodiments, the heterologous promoter is inserted 1-10,000, 10-1,000, 10-100, 10-5,000, 20-4,000, 30-3,000, 40-2,000, 50-1,000, 60-750, 70-500, 80-400, 90-300, 100-200, 1-1,000, 1,000-2,000, 2,000-3,000, 3,000-4,000, 4,000-5,000, 5,000-6,000, 6,000-7,000, 7,000-8,000, 8,000-9,000, or 9,000-10,000 nucleotides upstream from the first coding exon of the FOXP3 coding sequence. In some embodiments, the heterologous promoter is inserted into the first coding exon, such that a synthetic first coding exon is created, where the synthetic first coding exon differs from the endogenous first coding exon but still comprises a start codon that is in-frame with the FOXP3 coding sequence of downstream FOXP3 exons. In some embodiments, the heterologous promoter is inserted into the TSDR, such that the TSDR is modified and does not inhibit transcription of the endogenous FOXP3 coding sequence in inflammatory conditions.

In some embodiments, the nucleic acid comprising a heterologous promoter is comprised on a vector. In some embodiments, the vector is a viral vector. In some embodiments, the viral vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector.

In some embodiments, a nucleic acid comprising a promoter operably linked to a nucleic acid sequence encoding FoxP3 or a functional derivative thereof is introduced into the cell. Expression of a heterologous promoter and sequence encoding FoxP3 is useful, for example, for expressing functional FoxP3 in cells containing genomic mutations in the FOXP3 coding sequence (e.g., cells from subjects having IPEX syndrome). Additionally, additional coding sequences (e.g., encoding a CAR described herein) may be included in a nucleic acid, such that the heterologous promoter controls transcription of RNA encoding FoxP3 sequence and one or more other proteins (e.g., CAR). In some embodiments, the sequence encoding FoxP3 is a cDNA sequence that does not comprise an intron.

The introduced nucleic acid may be integrated into the genome at a targeted locus (e.g., by homologous recombination), integrated in a non-targeted manner (e.g., by delivery on a lentiviral vector), or not integrated. In some embodiments, the nucleic acid comprises a 5′ homology arm that is upstream from the promoter, and a 3′ homology arm that is downstream from the nucleic acid sequence encoding FoxP3, and both homology arms have homology to a targeted locus in a genome. Such homology arms promote insertion of the nucleic acid into the genome at the targeted locus by homologous recombination. The homology arms may be the same length, have similar lengths (within 100 bp of each other), or different lengths. In some embodiments, one or both homology arms have a length of 200-2,000 bp, 400-1,500 bp, 500-1,000 bp. In some embodiments, one or both homology arms are about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 bp, or about 2,000 bp.

In some embodiments, the nucleic acid is integrated at a FOXP3 locus in the genome. In some embodiments, the nucleic acid is integrated at a non-FOXP3 locus. In some embodiments, the targeted locus is a safe harbor locus. In some embodiments, the safe harbor locus is an AAVSI locus, a HIPP11 locus, or a ROSA26 locus. In some embodiments, the nucleic acid is integrated at a TCRα (TRAC) locus. In some embodiments, the nucleic acid is integrated at a TCRβ (TRBC) locus.

In some embodiments, a nuclease capable of cleaving the genome at a targeted locus, or a nucleic acid encoding the nuclease (e.g., an mRNA) is introduced into the cell. Following delivery of the nuclease or transcription of the nuclease inside the cell, the nuclease introduces a double-stranded break at the targeted locus, thereby promoting integration of a donor template (e.g., nucleic acid comprising a promoter and sequence encoding FoxP3, or nucleic acid comprising a heterologous promoter for promoting transcription of an endogenous FOXP3 coding sequence) into the genome at the targeted locus by homology-directed repair. The nuclease may be any nuclease known in the art, including a meganuclease, zinc finger nuclease, TALEN, or RNA-guided nuclease. In embodiments where an RNA-guided nuclease (or nucleic acid encoding an RNA-guided nuclease) is delivered, a guide RNA (or nucleic acid encoding a guide RNA) comprising a spacer sequence complementary to a genomic sequence at the targeted locus is introduced into the cell. A gRNA or nucleic acid encoding a gRNA may be introduced into the cell with the nuclease or nucleic acid encoding the nuclease, or introduced separately (e.g., in a separate vector or delivery vehicle). The RNA-guided nuclease may be any RNA-guided nuclease known in the art or described herein in the section entitled “Nucleases.”

In some embodiments, a nucleic acid comprising a heterologous promoter operably linked to a sequence encoding FoxP3 or a functional derivative thereof is present on a vector. In some embodiments, the vector is a viral vector. In some embodiments, the vector is a lentiviral vector. In some embodiments, the vector is an adeno-associated virus (AAV) vector. In some embodiments, the AAV vector is an AAV1, AAV2, AAV3, AAV4, AAV5, AAV6, AAV7, AAV8, AAV9, AAV10, or AAV11 vector. In some embodiments, the AAV vector is an AAV5 vector. In some embodiments, the AAV vector is an AAV6 vector. In some embodiments, the vector is a plasmid. In some embodiments, the vector is bacterial artificial chromosome. In some embodiments, the vector is human artificial chromosome. In some embodiments, the vector integrates into a chromosome of the genome, and RNA encoding FoxP3 is transcribed from the genome of the cell. In other embodiments, the vector does not integrate into a chromosome, and the sequence encoding FoxP3 is expressed episomally.

The heterologous promoter inserted into the FOXP3 locus or operably linked to the FOXP3 coding sequence may be any promoter known in the art. In some embodiments, the promoter is a constitutive promoter. In some embodiments, the promoter is an MND, PGK, or EF-la promoter. In some embodiments, the promoter is an MND promoter. In some embodiments, the promoter is an inducible promoter. In some embodiments, the promoter is inducible by a drug or steroid.

### Chemically Induced Signaling Complex (CISC)

Some embodiments of the methods of modifying cells provided herein comprise introducing into the cell one or more nucleic acids that collectively comprise (1) a first nucleic acid sequence encoding a first chemically inducible signaling complex (CISC) component, and (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in the cell. Similarly, some embodiments of cells described herein comprise (1) a first nucleic acid sequence encoding a first chemically inducible signaling complex (CISC) component, and (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in the cell. Additionally, some nucleic acids and vectors provided herein comprise (1) a first nucleic acid sequence encoding a first chemically inducible signaling complex (CISC) component, and/or (2) a second nucleic acid sequence encoding a second chemically inducible signaling complex (CISC) component, each CISC component comprising (a) an extracellular binding domain that is capable of binding to a CISC inducer molecule, (b) a transmembrane domain, and (c) an intracellular signaling domain, such that binding of the first and second CISC components to the CISC inducer molecule results in dimerization of the CISC components and a signal transduction event in a cell.

Expression of CISC components in a cell allows selective induction of signaling in a cell by manipulation of the presence and/or concentration of the CISC inducer molecule. Such controllable induction of signaling allows, for example, selective expansion of cells expressing both CISC components, where the signal transduction event results in proliferation of the cell. In some embodiments, where two nucleic acids, each encoding a different CISC component, are introduced into the cell, such selective expansion allows for selection of cells that contain both nucleic acids, as contacting a cell comprising only one CISC component would not induce dimerization with the absent second CISC component.

Non-limiting examples of intracellular signaling domains include IL-2Rβ and IL-2Rγ intracellular domains and functional derivatives thereof. In some embodiments, an intracellular signaling domain of one CISC component comprises an IL-2Rβ intracellular domain or a functional derivative thereof, and an intracellular signaling domain of the other CISC component comprises an IL-2Rγ domain or a functional derivative thereof. In some embodiments, dimerization of the CISC components induces phosphorylation of JAK1, JAK3, and/or STAT5 in the cell. In some embodiments, dimerization of the CISC components induces proliferation of the cell.

Non-limiting examples of transmembrane domains include IL-2Rβ, IL-2Rγ, erythropoietin (Epo), and thrombopoietin (Tpo) transmembrane domains. In some embodiments, the transmembrane domain of a CISC component is derived from the same protein as the intracellular signaling domain of the CISC component (e.g., a CISC component comprising an IL-2Rβ intracellular domain comprises an IL-2Rβ transmembrane domain). In some embodiments, one CISC component comprises an IL-2Rβ transmembrane domain, and the other CISC component comprises an IL-2Rγ transmembrane domain.

Non-limiting examples of extracellular binding domains capable of binding a CISC inducer molecule include an FK506-binding protein (FKBP) domain and an FKBP-rapamycin-binding (FRB) domain. FKBP and FRB domains are capable of binding to rapamycin or rapalogs, such as those described below. In some embodiments, an extracellular binding domain of one CISC component comprises an FKBP domain, and an extracellular binding domain of the other CISC component comprises an FRB domain. In some embodiments, the CISC components form a heterodimer in the presence of the CISC inducer molecule.

Each of the extracellular binding domains, transmembrane domains, and intracellular signaling domains of the CISC components described herein may be connected to another domain of the same CISC component by a linker. Linkers are known in the art. In some embodiments, the linker comprises 1, 2, 3, 4, 5, 6, 7, 8, 9 or 10 amino acids, such as glycines, or a number of amino acids, such as glycine, within a range defined by any two of the aforementioned numbers. In some embodiments, the glycine spacer comprises at least 3 glycines.

An extracellular binding domain may be connected to a transmembrane domain by a hinge domain. A hinge refers to a domain that links the extracellular binding domain to the transmembrane domain and may confer flexibility to the extracellular binding domain. In some embodiments, the hinge domain positions the extracellular domain close to the plasma membrane to minimize the potential for recognition by antibodies or binding fragments thereof. In some embodiments, the extracellular binding domain is located N-terminal to the hinge domain. In some embodiments, the hinge domain may be natural or synthetic.

In some embodiments, the CISC inducer molecule is rapamycin or a rapalog. In some embodiments, the CISC inducer molecule is rapamycin. Non-limiting examples of rapalogs include everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and metabolites or derivatives thereof.

In some embodiments, a method comprises introducing into a cell a nucleic acid encoding a third CISC component that is capable of binding to the CISC inducer molecule. Such CISC components are useful, for example, for binding to the intracellular CISC inducer molecules (e.g., intracellular rapamycin), thereby preventing the bound CISC inducer molecule from interacting with other intracellular molecules or structures (e.g., preventing rapamycin from interacting with mTOR). In some embodiments, the third CISC component is a soluble protein that does not comprise a transmembrane domain. In some embodiments, the third CISC component comprises an intracellular FRB domain. In some embodiments, a third CISC component is a soluble protein comprising an FRB domain and lacking a transmembrane domain.

Nucleic acids encoding a first, second, and/or third CISC component may be comprised in one or more vectors. In some embodiments, a nucleic acid encoding a first CISC component is present on a separate vector from a nucleic acid encoding the second CISC component. In some embodiments, a nucleic acid encoding the third CISC component is present on the same vector as a nucleic acid encoding the first or second CISC component. In other embodiments, a nucleic acid encoding the third CISC component is present on a distinct vector from nucleic acids encoding the first and/or second CISC components. In some embodiments, one or more vectors are viral vectors. In some embodiments, one or more vectors are lentiviral vectors. In some embodiments, one or more vectors are adeno-associated viral (AAV) vectors. In some embodiments, one or more AAV vectors is an AAV1, AAV2, AAV3, AAV4, AAV5, AAV6, AAV7, AAV8, AAV9, AAV10, or AAV11 vector. In some embodiments, one or more AAV vectors are AAV5 vectors. In some embodiments, one or more AAV vectors are AAV6 vectors.

In some embodiments, one or more CISC components further comprise a signal peptide. The signal peptide may be any signal peptide known in the art that directs the translated CISC component to the cell membrane.

### Pharmaceutical Compositions

Some aspects of the disclosure relate to a pharmaceutical composition comprising a cell, vector, or nucleic acid described herein, and a pharmaceutically acceptable excipient or carrier. Such pharmaceutical compositions are formulated, for example, for systemic administration, or administration to target tissues (e.g., for site-specific activation). “Acceptable” means that the excipient (carrier) must be compatible with the active ingredient of the composition (and preferably, capable of stabilizing the active ingredient) and not deleterious to the subject to be treated. Pharmaceutically acceptable excipients, carriers, buffers, stabilizers, isotonicizing agents, preservatives or antioxidants, or other materials well known to those skilled in the art. Such materials should be non-toxic and should not interfere with the efficacy of the active ingredient. The precise nature of the carrier or other material may depend on the route of administration, e.g., parenteral, intramuscular, intradermal, sublingual, buccal, ocular, intranasal, subcutaneous, intrathecal, intratumoral, oral, vaginal, or rectal. See, e.g., Remington: The Science and Practice of Pharmacy 20th Ed. (2000) Lippincott Williams and Wilkins, Ed. K. E. Hoover. The pharmaceutical compositions to be used for in vivo administration must be sterile, with the exception of any cells, viruses, and/or viral vectors being used to achieve a biological effect (e.g., immunosuppression). This is readily accomplished by, for example, filtration through sterile filtration membranes. The pharmaceutical compositions described herein may be placed into a container having a sterile access port, for example, an intravenous solution bag or vial having a stopper pierceable by a hypodermic injection needle.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical, ingestible, and intradermal routes. Administration can be systemic or local. In certain embodiments, the pharmaceutical composition is adapted for oral administration. In some embodiments, the pharmaceutical composition comprising the non-degradable engineered polymeric antigen and the pharmaceutical composition comprising the engineered immune cell (e.g., CAR Treg cell) are adapted for different routes of administration. For example, in some embodiments, the pharmaceutical composition comprising the non-degradable engineered polymeric antigen is adapted for oral administration, while the pharmaceutical composition comprising the engineered immune cell is adapted for intravenous or intramuscular administration.

The pharmaceutical compositions described herein to be used in the present methods can comprise pharmaceutically acceptable carriers, buffer agents, excipients, salts, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, the pharmaceutical composition described herein comprises lipid nanoparticles which can be prepared by methods known in the art, such as described in Epstein et al., Proc Natl Acad Sci USA . 1985. 82: 3688; Hwang et al. Proc Natl Acad Sci USA. 1980. 77:4030; and U.S. Pat. Nos. 4,485,045 and 4,544,545. Lipids used in the formulation of lipid nanoparticles for delivering nucleic acids are generally known in the art, and include ionizable amino lipids, non-cationic lipids, sterols, and polyethylene glycol-modified lipids. See, e.g., Buschmann et al., Vaccines. 2021. 9(1): 65. In some embodiments, the nucleic acid is surrounded by the lipids of the lipid nanoparticle and present in the interior of the lipid nanoparticle. In some embodiments, the nucleic acid is dispersed throughout the lipids of the lipid nanoparticle. In some embodiments, the lipid nanoparticle comprises an ionizable amino lipid, a non-cationic lipid, a sterol, and/or a polyethylene glycol (PEG)-modified lipid.

Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE).

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device, or the nebulizing device may be attached to a face mask, tent or intermittent positive pressure breathing machine. Solution, suspension, or powder compositions may be administered, preferably orally or nasally, from devices which deliver the formulation in an appropriate manner.

The pharmaceutical compositions described herein can include a therapeutically effective amount of any cell, vector, and/or nucleic acid described herein. For example, in some embodiments, the pharmaceutical composition includes a cell, vector, or nucleic acid at any of the doses described herein.

A “therapeutically effective amount” refers to an amount effective, at dosages and for periods of time necessary, to achieve a desired therapeutic result. The therapeutically effective amount may vary according to factors such as the age, sex, and weight of the individual, and the ability of the cell, nucleic acid, or vector to affect a desired response in the subject.

Pharmaceutical compositions can be prepared in accordance with known techniques. See, e.g., Remington, The Science And Practice of Pharmacy (21st ed., Philadelphia, Lippincott, Williams & Wilkins, 2005). For example, cells, vectors, or nucleic acids described herein may be admixed with a pharmaceutically acceptable excipient, and the resulting composition is administered to a subject. The carrier must be acceptable in the sense of being compatible with any other ingredients in the formulation and must not be deleterious to the subject. The carrier can be a solid or a liquid, or both, and can be formulated with the compound as a unit-dose formulation.

In some embodiments, a pharmaceutical composition comprises cells (e.g., engineered immune cells) at a dose of about 10⁴ to about 10¹⁰ cells/kg. In some embodiments, the pharmaceutical composition comprises cells at a dose of about: 10⁴ to 10⁵, 1⁰⁵ to 10⁶, 10⁶ to 10⁷, 10⁷ to 10⁸, 10⁸ to 10⁹, or 10⁹ to 10¹⁰ cells/kg. In some embodiments, a pharmaceutical composition comprises cells at a dose of about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.6×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶,6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷,or 1.0×10⁸ cells/kg.

In some embodiments, rapamycin or a rapalog is administered to the subject before the administration of cells, in conjunction with cells, and/or following the administration of cells. Administration of rapamycin or a rapalog that is capable of inducing dimerization of the CISC components on the surface of a cell results in continued IL-2 signal transduction in vivo, promoting survival and proliferation of the CISC-expressing cell without the undesired effects that would be caused by IL-2 administration, such as activation of other T cells. In some embodiments, the rapamycin or rapalog that is administered is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, or a metabolite or derivative thereof. In some embodiments, the rapamycin or rapalog is administered at a dose of 0.001 mg/kg to 10 mg/kg body mass of the subject, or a dose between 0.001 mg/kg and 10 mg/kg. In some embodiments, the rapamycin or rapalog is administered at a dose of 0.001 mg/kg to 0.01 mg/kg, 0.01 mg/kg to 0.1 mg/kg, 0.1 mg/kg to 1 mg/kg, or 1 mg/kg to 10 mg/kg. In some embodiments, rapamycin or rapalog is administered at a dose of 0.01 nM, 0.02 nM, 0.03 nM, 0.04 nM, 0.05 nM, 0.06 nM, 0.07 nM, 0.08 nM, 0.09 nM, 0.1 nM, 0.2 nM, 0.3nM, 0.4 nM,0.5 nM, 0.6 nM, 0.7 nM, 0.8 nM, 0.9 nM, 1.0 nM, 1.5 nM, 2.0 nM, 2.5 nM, 3.0 nM, 3.5 nM, 4.0nM, 4.5 nM, 5.0 nM, 5.5 nM, 6.0 nM, 6.5 nM, 7.0 nM, 7.5 nM, 8.0 nM, 8.5 nM, 9.0 nM, 9.5 nM, 10 nM, 11 nM, 12 nM, 13 nM, 14 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, or 100 nM or a concentration within a range defined by any two of the aforementioned values.

In some embodiments, a pharmaceutical composition comprises an effective amount of a vector or nucleic acid (e.g., CAR) described herein. In some examples, the pharmaceutical composition comprises about 0.1 mg/kg to about 3 mg/kg of the vector or nucleic acid. In some embodiments, the pharmaceutical composition comprises about 0.1 mg/kg, about 0.25 mg/kg, about 0.5 mg/kg, about 0.75 mg/kg, about 1.0 mg/kg, about 1.5 mg/kg, about 2.0 mg/kg, about 2.5 mg/kg, or about 3.0 mg/kg of the vector or nucleic acid. In some embodiments, pharmaceutical composition comprises about 0.1 mg/kg to about 0.25 mg/kg, about 0.25 mg/kg to about 0.5 mg/kg, about 0.5 mg/kg to about 0.75 mg/kg, about 0.75 mg/kg to about 1.0 mg/kg, about 1.0 mg/kg to about 1.5 mg/kg, about 1.5 mg/kg to about 2.0 mg/kg, about 2.0 mg/kg to about 2.5 mg/kg, or about 2.5 mg/kg to about 3.0 mg/kg of the vector or nucleic acid.

### Methods of Use

Some aspects of the disclosure relate to methods of administering a cell, vector, nucleic acid as well as a non-degradable engineered polymeric antigen described herein to a subject. Provided herein is a method comprising administering to a subject any one of the therapeutic cells capable of evading host immune attack, as disclosed herein, e.g., to treat a disease or condition in the subject. In some embodiments, a subject is a mammal. In some embodiments, the subject is a human.

In some embodiments, “administering” or “administration” means providing a material to a subject in a manner that is pharmacologically useful. To “treat” a disease as the term is used herein, means to reduce the frequency or severity of at least one sign or symptom of a disease or disorder experienced by a subject. The compositions described above or elsewhere herein are typically administered to a subject in an effective amount, that is, an amount capable of producing a desirable result. An effective amount, in some embodiments, comprises 10³-10¹² cells (e.g., 10³-10¹², 10³-10¹¹, 10³-10¹⁰, 10³-10⁹, 10³-10⁸, 10³-10⁷, 10³-10⁶, 10³-10⁵, 10³-10⁴, 10⁴-10¹², 10⁴-10¹¹, 10⁴-10¹⁰, 10⁴-10⁹, 10⁴-10⁸, 10⁴-10⁷, 10⁴-10⁶, 10⁴-10⁵, 10⁵-10¹², 10⁵-10¹¹, 10⁵-10¹⁰, 10⁵-10⁹, 10⁵-10⁸, 10⁵-10⁷, 10⁵-10⁶, 10⁶-10¹², 10⁶-10¹¹, 10⁶-10¹⁰, 10⁶-10⁹, 10⁶-10⁸, 10⁶-10⁷, 10⁷-10¹², 10⁷-10¹¹, 10⁷-10¹⁰, 10⁷-10⁹, 10⁷-10⁸, or any discrete number encompassed therein), and may be administered to a subject in a single dose, or over multiple doses (e.g., 2, 3, 4, 5, or more) doses, either simultaneously or sequentially. In some embodiments, the number of therapeutic cells administered to a subject depends upon the anatomical location, administration route, cell source, patient age and medical history, indication, disease severity, and other factors.

In some embodiments, the non-degradable engineered polymeric antigen is administered concomitantly or after administration of a first dose of the engineered cells. In some embodiments, the non-degradable engineered polymeric antigen is administered 1, 2, 3, 4, 5, 6, 7 or more days before a first dose of engineered cells. In some embodiments, the engineered cells are administered daily (e.g., for 1, 2, 3, 4, 5, 6, 7, or more days) or weekly (e.g., for 1, 2, 3, 4, 5, 6, 7, 8, 9, 10, 11, 12 or more weeks), or monthly (e.g., for 1, 2, 3, 4, 5, 6, 7, 8, 9, 10, 11, 12 or more months). In some embodiments, the non-degradable engineered polymeric antigen is administered daily (e.g., for 1, 2, 3, 4, 5, 6, 7, or more days) or weekly (e.g., for 1, 2, 3, 4, 5, 6, 7,8, 9, 10, 11, 12 or more weeks), or monthly (e.g., for 1, 2, 3, 4, 5, 6, 7, 8, 9, 10, 11, 12 or more months). In some embodiments, the non-degradable engineered polymeric antigen is administered daily after the first dose of engineered T cells. In some embodiments, the non-degradable engineered polymeric antigen is administered every other day after the first dose of engineered T cells. In some embodiments, the non-degradable engineered polymeric antigen is administered weekly after the first dose of engineered T cells. In some embodiments the non-degradable engineered polymeric antigen is administered every other week after the first dose of engineered T cells. In some embodiments, the non-degradable engineered polymeric antigen is administered monthly after the first dose of engineered T cells. In some embodiments, the non-degradable engineered polymeric antigen is administered every other month after the first dose of engineered T cells. In some embodiments, the non-degradable engineered polymeric antigen is administered concomitantly or after administration of a first dose of the engineered cells in response to at least one symptom. In some embodiments, in any of the methods described herein, the non-degradable engineered polymeric antigen may be administered until the subject is no longer suitable for receiving treatment. In some embodiments, in any of the methods described herein, the non-degradable engineered polymeric antigen may be administered for the remainder of the subject's lifetime.

In some embodiments, the non-degradable engineered polymeric antigen is administered in an effective amount, e.g., a therapeutically effective amount, such as 0.001 mg/kg to 10 mg/kg body mass of the subject, or a dose between 0.001 mg/kg and 10 mg/kg. In some embodiments, a dose of 0.001 mg/kg to 0.01 mg/kg, 0.01 mg/kg to 0.1 mg/kg, 0.1 mg/kg to 1 mg/kg, 1 mg/kg to 10 mg/kg, or 10 mg/kg or 100 mg/kg of non-degradable engineered polymeric antigen is administered. In some embodiments a therapeutically effective amount is less then 17 g of non-degradable engineered polymeric antigen. In some embodiments a therapeutically effective amount is between 0.1-1 g, 1-2, 1-3, 1-4,-1-5, 1-6, 1-7, 1-8, 1-9, 1-10, 1-11, 1-12, 1-13, 1-14, 1-15, 1-16, 1-17, 2-3, 2-4, 2-5, 2-6, 2-7, 2-8. 2-9. 2-10, 2-11, 2-12, 2-13, 2-14, 2-15, 2-16, 2-17, 3-4, 3-5, 3-6, 3-7, 3-8, 3-9, 3-10, 3-11, 3-12, 3-13, 3-14, 3-15, 3-16, 3-17, 4-5,4-6, 4-7, 4,8-, 4-9, 4-10, 4-11, 4-12, 4-13, 4-14, 4-15, 4-16, 4-17, 5-6, 5-7, 5-8, 5-9, 5-10, 5-11, 5-12, 5-13, 5-14, 5-15, 5-16, 5-17, 6-7, 6-8, 6-9, 6-10, 6-11, 6-12, 6-13, 6-14, 6-15, 6-16, 6-17, 7-8, 7-9, 7-10, 7-11, 7-12, 7-13, 7-14, 7-15, 7-16, 7-17, 8-9, 8-10, 8-11, 8-12, 8-13, 8-14, 8-15, 8-16, 8-17, 9-10, 9-11, 9-12, 9-13, 9-14, 9-15, 9-16, 9-17, 10-11, 10-12, 10-13, 10-14, 10-15, 10-16, 10-17, 11-12, 11-13, 11-14, 11-15, 11-16, 11-17, 12-13, 12-14, 12-15, 12-16, 12-17, 13-14, 13-15, 13-16, 13-17, 14-15, 14-16, 14-17, 15-16, 15-17, 16-17 grams of non-degradable engineered polymeric antigen. In some embodiments, a therapeutically effective amount is 1, 2, 3, 4, 5, 6, 7, 8, 9, 10, 11, 12, 13, 14, 15, 16, or 17 grams of non-degradable engineered polymeric antigen.

The therapeutically effective amount is typically determined in appropriately designed clinical trials (dose range studies) and the person skilled in the art will know how to properly conduct such trials to determine the effective amount. In some embodiments, a therapeutically effective amount of the non-degradable engineered polymeric antigen is an amount that is not absorbed into the gastrointestinal tract barrier in the absence of a breach of the intestinal tract barrier (e.g., leaky places in the mucosal gut barrier) (i.e., an amount that is absorbed into the gastrointestinal tract barrier during inflammation). In some embodiments, a therapeutically acceptable amount of the non-degradable engineered polymeric antigen is not absorbed into the blood stream in the absence of a breach of the intestinal tract barrier (e.g., leaky places in the mucosal gut barrier).

In some embodiments, the non-degradable engineered polymeric antigen is administered in a separate pharmaceutical composition from the engineered cells. In some embodiments, the non-degradable engineered polymeric antigen pharmaceutical composition and the engineered immune cell composition are administered 1, 2, 3, 4, 5, 6, 7, 8, or more times per day for 1, 2, 3, 4, 5, 6, 7, 8, 9, 10, 11, 12, 13, or 14 or more days. In some embodiments, the non-degradable engineered polymeric antigen pharmaceutical composition and the engineered immune cell composition are administered for 1, 2, 3, 4, 5, 6, 7, 8, 9, 10, 11, 12, 13, 14, 15, 16, 17, 18, 19, or 20 or more weeks. In some embodiments the non-degradable engineered polymeric antigen pharmaceutical composition is administered daily, every other day, twice a week, weekly, biweekly, every other week, monthly, bimonthly, every other month, every three months, or every four months. In some embodiments the engineered immune cell pharmaceutical composition is administered daily, every other day, twice a week, weekly, biweekly, every other week, monthly, bimonthly, every other month, every three months, or every four months.

In some embodiments, administration is site-specific. Without wishing to be bound by theory, it is thought that the non-degradable engineered polymeric antigen will diffuse through permeable tissue (e.g., inflamed areas of a GI tract), where it will activate the engineered immune cells, leading to a site-specific immunosuppression. In some embodiments, the site is the gastrointestinal tract, one or more joints, a visceral organ, one or more muscles, the skin, the brain, the spine, or the cerebrospinal fluid. In some embodiments, the site of the site-specific activation is the GI tract. In some embodiments, administration will occur systemically. In some embodiments, systemic administration occurs intravenously or interperitoneally. In some embodiments administration will occur at multiple sites.

In some embodiments, a method comprises administering to the subject a cell that had previously been obtained from that subject before being administered (i.e., the cell is an autologous cell). In some embodiments, a method comprises (i) isolation of cells from a subject; (ii) processing the cells by any method (e.g., gene editing or introducing a vector) described herein; and (iii) administering the processed cells to the same subject. In some embodiments, a method comprises administering to the subject a cell that had previously been obtained from a different subject than the one to whom the cell is administered (i.e., the cell is an allogeneic cell). In some embodiments, a method comprises (i) isolation of cells from a first subject; (ii) processing the cells by any method (e.g., gene editing or introducing a vector) described herein; and (iii) administering the processed cells to a second subject.

Some embodiments of the methods, cells, systems, and compositions described herein include any of the cells, vectors, nucleic acids, or lipid nanoparticles described herein, for use as a medicament. In some embodiments, the cell, vector, nucleic acid, or lipid nanoparticle is for use in a method of preventing, treating, inhibiting, or ameliorating an inflammatory, autoimmune, or allergic condition or disease in a subject.

In some embodiments, a cell is described herein for use in a method of preventing, treating, inhibiting, or ameliorating an inflammatory, autoimmune, or allergic condition or disease in a subject. In some embodiments, the cell is autologous to the subject (i.e., derived from the subject). In other embodiments, the cell is allogeneic to the subject (i.e., derived from a different subject).

In some embodiments, the subject has an autoimmune condition, an allergic condition, and/or an inflammatory condition. In some embodiments, the subject is suspected of having autoimmune condition, an allergic condition, and/or an inflammatory condition. In some embodiments, the subject may develop an autoimmune condition, an allergic condition, and/or an inflammatory condition.

In some embodiments, the autoimmune disease or condition is eczema, vitiligo, scleroderma, psoriasis, vasculitis, polymyalgia rheumatica, fibromyalgia, type 1 diabetes mellitus, multiple sclerosis, lupus, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, bullous pemphigoid, pemphigus vulgaris, autoimmune hepatitis, Sjogren's syndrome, celiac disease, central nervous system vasculitis, autoimmune-related epilepsy, Hashimoto's encephalopathy, steroid-responsive encephalopathy, neuromyelitis optica, optic neuritis, neurosarcoidosis, or neuro-Behcet's disease.

In some embodiments, the allergic condition is allergic asthma, atopic dermatitis, pollen allergy, food allergy, drug hypersensitivity, or contact dermatitis.

In some embodiments, the inflammatory condition is pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome (ARDS), uveitis, inflammatory bowel disease (IBD), ulcerative colitis, graft-versus-host disease (GVHD), tolerance induction for transplantation, transplant rejection, or sepsis. In some embodiments, the subject has or is at risk of developing inflammatory bowel disease. In some embodiments, the subject has or is at risk of developing Crohn's disease. In some embodiments, the subject has or is at risk of developing inflammatory colitis. In some embodiments, the subject has or is at risk of developing rheumatoid arthritis.

In some embodiments, the subject has at least 1 inflammatory or autoimmune disease. In some embodiments, the subject has at least 2 inflammatory and/or autoimmune diseases. In some embodiments, the subject has at least 3 inflammatory and/or autoimmune diseases. In some embodiments, the subject has at least 4 inflammatory and/or autoimmune diseases. In some embodiments, the subject has at least 5 inflammatory and/or autoimmune diseases.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean ±10% of the recited numerical value.

Where a range of values is provided, each value between and including the upper and lower ends of the range are specifically contemplated and described herein.

1. A method for activation of engineered regulatory T cells present in a gastrointestinal (GI) tract of a subject, the method comprising administering to said subject said engineered regulatory T cells, and orally administering to the subject a composition comprising a non-degradable engineered polymeric antigen, wherein the engineered T cells comprise a chimeric antigen receptor (CAR) specific to the antigen.

2. (canceled)

3. (canceled)

4. (canceled)

5. (canceled)

6. (canceled)

7. The method of claim 1, wherein the subject has GI tract permeability wherein the non-degradable engineered polymeric antigen is absorbed into the gastrointestinal tract barrier at a site of breach or a site of local increase in permeability but does not get absorbed into the blood stream.

8. (canceled)

9. The method of claim 1, wherein the non-degradable engineered polymeric antigen comprises a polyethylene glycol (PEG), dextran, ethylene glycol, polyethylene oxide, poly(¹-caprolactone) (PCL), polyglycolic acid (PGA), polylactic acid (PLA), and their copolymer poly(glycolic acid) (PLGA), polyvinyl alcohol (PVA), polyurethane (PU), collagen, fibrin, fibrinogen, gelatin, silk, elastin, myosin, keratin, actin, chitin, chitosan, alginate, hyaluronic acid, cellulose, agarose, or glycosaminoglycan.

10. The method of claim 9, wherein the non-degradable engineered polymeric antigen comprises a polyethylene glycol (PEG).

11. The method of claim 9, wherein the non-degradable engineered polymeric antigen comprises dextran.

12. The method of claim 1, wherein the engineered polymeric antigen has a molecular weight of 400-10,000 g/mol.

13. The method of any one of claim 1, wherein the subject has a disease selected from an inflammatory or autoimmune GI disease.

14. (canceled)

15. (canceled)

16. The disease of claim 13, wherein the disease is inflammatory bowel disease, Crohn's disease, or colitis.

17. (canceled)

18. (canceled)

19. (canceled)

20. (canceled)

21. The method of claim 1, wherein the regulatory T cells are engineered to stably express FoxP3, IL10, or IL2.

22. (canceled)

23. (canceled)

24. (canceled)

25. (canceled)

26. The method of claim 1, wherein the CAR comprises a signaling domain comprises the domain of CD3zeta and the co-stimulatory domain comprises the domain of CD28.

27. The method of claim 1, wherein the CAR comprises a signaling domain comprises the domain of CD3zeta and the co-stimulatory domain comprises the domain of CD137.

28. The method of claim 10, wherein
the administering of the non degradable engineered polymeric antigen is through oral ingestion, the non-degradable engineered polymeric antigen is a PEG having a molecular weight of 400-10,000 g/mol.

29. (canceled)

30. (canceled)

31. (canceled)

32. (canceled)

33. (canceled)

34. (canceled)

35. A non-degradable engineered polymeric antigen, wherein:
a. the non-degradable engineered polymeric antigen is a polyethylene glycol (PEG) or dextran,
b. the non-degradable engineered polymeric antigen is formulated in an ingestible form, and
c. the non-degradable engineered polymeric antigen is engineered to have a molecular weight between 200 and 50,000 g/mol.

36. (canceled)

37. (canceled)

38. (canceled)

39. (canceled)

40. (canceled)

41. (canceled)

42. (canceled)

43. (canceled)

44. (canceled)

45. (canceled)

46. A method of producing an engineered regulatory T cell comprising a chimeric antigen receptor (CAR), the method comprising:
a. providing a nucleic acid molecule encoding a CAR comprising an extracellular antigen binding domain comprising a sequence that binds to a non-degradable engineered polymeric antigen, a signaling domain of CD3zeta and a costimulatory domain of CD28 or CD137;
b. providing a regulatory T cell; and
c. introducing said provided nucleic acid molecule into said provided regulatory T cell.

47. (canceled)

48. (canceled)

49. (canceled)

50. The method of claim 46, wherein at least one of:
a. the regulatory T cell is obtained from a donor; and
b. the regulatory cell is obtained from an individual having an autoimmune GI disease.

51. (canceled)

52. (canceled)

53. (canceled)

54. The method of claim 46, wherein the non-degradable engineered polymeric antigen is a polyethylene glycol or dextran.

55. (canceled)

56. (canceled)

57. (canceled)

58. The method of claim 46, wherein the nucleic acid molecule is introduced in the cell in vitro via a transfection, a transduction, a gene editing technique, a viral or non-viral vector, a nanoparticle or a transposon.

59. (canceled)

60. (canceled)

61. (canceled)

62. (canceled)

63. (canceled)

64. (canceled)

65. The method of claim 46, wherein the extracellular domain comprises a polypeptide comprising an antibody, antibody fragment, antigen-binding domain of an antibody (e.g., single chain antibodies, Fab and sFab fragments, F(ab′)2, Fd fragments, Fv fragments, scFv, CDRs, and domain antibody (dAb) fragments), immunoglobulin variable domain, or immunoglobulin variable domain sequence.

66. The method of claim 46, further comprising selecting a non-degradable engineered polymeric antigen that is of a size that it is absorbed into a GI tract barrier at a site of breach but is not absorbed into the blood stream through an intact GI tract barrier.

67. The method of claim 46, wherein providing a nucleic acid molecule comprises producing a plurality of CARs, testing binding of said plurality of CARs to said non-degradable engineered polymeric antigen and selecting a CAR that binds.
